(19) United States
(12) Patent Application Publication
(10) Pub. No.: US 2003/0059451 A1
(43) Pub. Date: Mar. 27, 2003

(54) Use of orally administered dehydroepiandrosterone, precursors and derivatives thereof in order to improve the papery aspect of the skin

(76) Inventors: Olivier De La Charriere, Paris (FR); Stephanie Nouveau, Boulogne Billancourt (FR); Francoise Forette, Paris (FR); Etienne Emile Baulieu, Neuilly Sur Seine (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/181,001

(22) PCT Filed: Jan. 10, 2001
(86) PCT No.: PCT/FR01/00063

(30) Foreign Application Priority Data
Jan. 12, 2000 (FR) 00 00322

(51) Int. Cl. A61K 31/56; A61K 7/00

(52) U.S. Cl. 424/401; 514/178; 514/26

(57) ABSTRACT

The use of at least one compound selected from the group consisting of dehydroepiandrosterone, the chemical precursors thereof, the chemical derivatives thereof, the biological precursors and metabolic derivatives thereof, in the production of a composition which can be administered orally and used in order to reduce or prevent the papery aspect of the skin.

The present invention relates to the use of dehydroepiandrosterone, of its precursors and of its derivatives, for producing a composition suitable for oral administration and intended to be used to reduce or prevent an increase in the weathered (papyrus-like) appearance of the skin.

During aging, one of the major criteria in the degradation of the appearance of the skin is the accentuation of its weathered appearance. The weathered or papyrus-like appearance is a sign of aging of the skin, characterized by a change in the visual appearance and also the touch behavior of the skin. Visually, the skin takes on the appearance of cigarette paper, giving it an appearance similar to that of a sheet of papyrus. When the skin is gently pinched between the thumb and index finger, the folding of the skin takes on a finely folded appearance with numerous sharp folds like creased paper. Finally, the feel of the skin shows that its superficial portions appear to be floating on the deep portions, giving the skin, at the very advanced stage of weathered (papyrus-like) appearance, a crumpled look.

To date, the only known active compound capable of preventing this increase in weathered or papyrus-like appearance is retinoic acid, which is a reference compound in the treatment of aging of the skin.

Thus, there remains a need for novel agents capable of reducing or preventing an increase in the weathered (papyrus-like) appearance of the skin.

Now, the applicant has found, unexpectedly, that dehydroepiandrosterone (DHEA), or at least one of its chemical precursors, of its chemical derivatives, of its biological precursors or of its metabolic derivatives, administered orally, is capable of reducing or preventing the weathered (papyrus-like) appearance of the skin.

DHEA is a natural steroid produced essentially by the adrenocortical glands. It is known for its antiaging properties related to its ability to promote epidermal keratinization (JP-07196467) and to combat osteoporosis (U.S. Pat. No. 5,824,671). It is used in the treatment of dry skin, due to its ability to increase the endogenous production and secretion of sebum and to reinforce the barrier effect of the skin (U.S. Pat. No. 4,496,556), and also in the treatment of obesity and of diabetes (WO 97/13500) and in various diseases of hormonal origin, such as some cancers or osteoporosis (WO 94/16709). It has also been proposed to use DHEA sulfate against alopecia (JP-60142908) and for treating the various signs of aging, such as elasticity, flaccidity or slacking of the skin (EP-0723775). However, none of those documents either describes or suggests the oral use of DHEA, of its chemical precursors, of its chemical derivatives, of its biological precursors or of its metabolic derivatives, for reducing or preventing an increase in the weathered (papyrus-like) appearance of the skin, which is the subject of the present invention.

Consequently, the subject of the present invention is the use of at least one compound chosen from the group consisting of dehydroepiandrosterone, its chemical precursors, its chemical derivatives, its biological precursors and its metabolic derivatives, for preparing a composition suitable for oral administration and intended to be used to reduce or prevent an increase in the weathered (papyrus-like) appearance of the skin.

In an advantageous embodiment of the use according to the invention, the biological precursor is chosen from the group comprising cholesterol, pregnenolone, 17α-hydroxypregnenolone, 5-androstenediol, dehydroepiandrosterone sulfate, 17α-hydroxypregnenolone sulfate and 5-androstenediol sulfate.

In another advantageous embodiment of this use, the chemical precursor is chosen from the group comprising sapogenins, such as, for example, diosgenin (or spirost-5-en-3-β-ol), and the natural extracts containing them, in particular fenugreek and extracts of Dioscoraceae such as wild yam.

In another advantageous embodiment of this use, the metabolic derivative is chosen from the group comprising 5-androstene-3β-17β-diol (adiol), 5-androstene-3β-17β-diol sulfate and 4-androstene-3,17-dione.

In another advantageous embodiment of this use, the chemical derivative is chosen from the group comprising the salts of DHEA, in particular the water-soluble salts of DHEA, such as for example DHEA sulfate, and the esters of DHEA, in particular DHEA salicylate.

In accordance with the use according to the invention, the composition may be a cosmetic composition or a dermatological composition, provided in all the pharmaceutical forms normally used for oral administration, in particular in the form of tablets which may or may not be breakable, of granules, of capsules, of gelatin capsules, of solutes, of suspensions or of solutions, and comprising at least one compound chosen from the group consisting of dehydroepiandrosterone, its precursors and its derivatives, as active principle, in combination with any suitable excipient.

In accordance with the use according to the present invention, the compounds are present in the composition in an amount which allows them to be administered at a dose of between 1 and 100 mg per day, preferably between 25 and 75 mg per day, said dosage being taken in one or more stages, for example with a unit dose of 50 mg.

The DHEA which can be used according to the invention is, for example, available from the company SIGMA or from the company AKZO NOBEL.

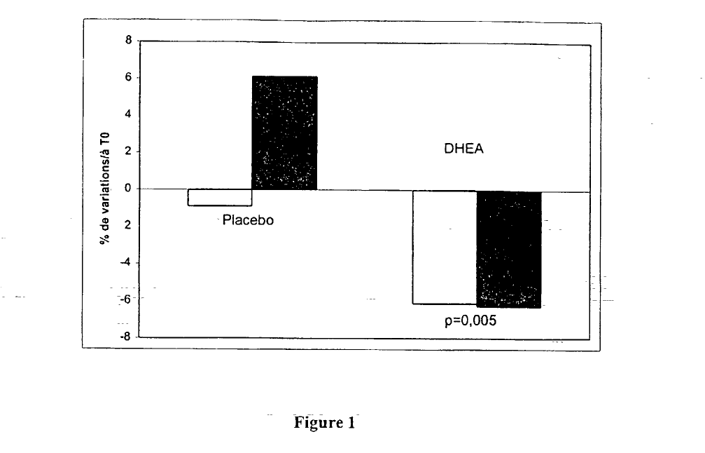

### Other characteristics and advantages of the invention appear in the remainder of the description and the examples illustrated in FIG. 1.

FIG. 1 illustrates the effect of DHEA on the weathered aspect of the skin, after 12 months of treatment at the dose of 50 mg/day orally, according to example 1; men (placebo n=15; DHEA=15); ▪ women (placebo n=17; DHEA n=16).

### EXAMPLE 1

### Evaluation of the Effect of DHEA Administered Orally on the Weathered Appearance of the Skin and on the Elasticity and Slackening of the Skin—Comparison with a Placebo

1. Methodology

1.1 Treatment

The evaluation was carried out using a randomized double-blind clinical study on 70 individuals (35 men and 35 women). The inclusion criteria were as follows: healthy volunteers, of both sexes, free of any severe pathological condition and 60 to 80 years old.

The treatment consisted, for 12 months, of a daily dose of 50 mg of DHEA, taken in the morning, in the form of a breakable tablet, for half the individuals, or as a placebo for the other half of the individuals.

1.2. Evaluation of the Weathered (Papyrus-Like) Appearance

The weathered (papyrus-like) appearance is evaluated clinically, blind, on the back of a hand at the start of treatment (T0) and at the end of treatment (T12), by the same clinician.

The weathered (papyrus-like) appearance is represented by a clinical score ranging from 0 (weathered appearance absent or only very slightly noticeable) to 9 (very marked weathered appearance).

The statistical analysis is performed using the Wilcoxon test.

1.3. Evaluation of the Elasticity:

The elasticity of the skin was evaluated with the Derma Torque Meter® (DTM® Dia-Stron Limited, Andover, Hampshire, United Kingdom). The biomechanical properties (elasticity) of the skin of the ventral side of the forearm is measured. The DTM® makes it possible to study the response of the skin to a deformation applied in the direction parallel to its surface. The rotation of a disk stuck to the skin is measured and the elasticity of the skin is thus evaluated, which is one of the best markers of aging (Escoffier C. et al. (1989), J. Invest. Dermatol., 93, 353-357).

The elasticity is expressed as the ratio of extensibility to recovery.

The statistical analysis is performed using the Wilcoxon test.

1.4. Evaluation of the Slackening of the Skin

The slackening of the skin is evaluated clinically, blind, on the ventral side of the forearm and on the skin of the face at the start of treatment (T0) and at the end of treatment (T12), by the same clinician. The slackening of the skin is represented by a clinical score ranging from 0 (slight or hardly noticeable slackening) to 9 (very marked slackening). The statistical analysis is performed using the Wilcoxon test.

2. Results:

2.1. Effects on the Weathered (Papyrus-Like) Appearance:

They are illustrated in FIG. 1.

When the variations in the weathered (papyrus-like) appearance are observed relative to the initial state at T0, an increase in the mean scores for this criterion is observed, for the group of women treated with the placebo, which reflects a considerable degradation of the appearance of the skin in one year.

On the contrary, the oral treatment with DHEA not only makes it possible to avoid this degradation (in the case of the group of men), but even to improve the weathered (papyrus-like) appearance (decrease in the mean scores in the women treated with DHEA). This variation is statistically significant relative to the placebo group (p=0.005).

These results show that, after oral administration, DHEA improves the weathered (papyrus-like) appearance of the skin.

2.2 Effects on the Elasticity and Slackening of the Skin:

They are illustrated in table 1.

TABLE 1

| | DHEA | | Placebo | | Significance |
|---|---|---|---|---|---|
| Parameter | T0 | T12 | T0 | T12 | (Wilcoxon test) |
| Slackening | 6.69 | 6.81 | 6.29 | 6.35 | P = 0.5 |
| of the skin | | | | | |
| of the face | | | | | |
| Slackening | 5.44 | 7.19 | 4.71 | 7 | P = 0.15 |
| of the skin | | | | | |
| of the fore- | | | | | |
| arm | | | | | |
| Elasticity | 0.36 | 0.31 | 0.34 | 0.38 | P = 0.55 |
| of the skin | | | | | |
| of the fore- | | | | | |
| arm | | | | | |

These results show that the decrease in the weathered (papyrus-like) appearance of the skin is not related to a variation in the elasticity or in the slackening of the skin, since these parameters do not significantly vary after treatment with DHEA administered orally.

1. The use of at least one compound chosen from the group consisting of dehydroepiandrosterone, its chemical precursors, its chemical derivatives, its biological precursors and its metabolic derivatives, for preparing a composition suitable for oral administration and intended to be used to reduce or prevent an increase in the weathered or papyrus-like appearance of the skin.

2. The use as claimed in claim 1, characterized in that the biological precursor is chosen from the group comprising cholesterol pregnenolone, 17α-hydroxypregnenolone, 5-androstenediol, dehydroepiandrosterone sulfate, 17α-hydroxypregnenolone sulfate and 5-androstenediol sulfate.

3. The use as claimed in claim 1, characterized in that the chemical precursor is chosen from the group consisting of sapogenins and the natural extracts containing them.

4. The use as claimed in claim 1, characterized in that the metabolic derivative is chosen from the group comprising 5-androstene-3β-17β-diol (adiol), 5-androstene-3β-17β-diol sulfate and 4-androstene-3,17-dione.

5. The use as claimed in claim 1, characterized in that the chemical derivative is chosen from the group consisting of the salts and the esters of DHEA.

6. The use as claimed in any one of claims 1 to 5, characterized in that the composition is a cosmetic composition.

7. The use as claimed in any one of claims 1 to 5, characterized in that the composition is a dermatological composition.

8. The use as claimed in any one of claims 1 to 7, characterized in that the compounds are present in the composition in an amount which allows them to be administered at a dose of between 1 and 100 mg per day, preferably between 25 and 75 mg per day.
